(19) United States
(12) Patent Application Publication
(10) Pub. No.: US 2019/0216823 A1
(43) Pub. Date: Jul. 18, 2019

(54) METHYLPREDNISOLONE PHARMACEUTICAL SUSPENSION

(71) Applicant: Hyloris Developments SA, Liège (BE)

(72) Inventors: Thomas Gerner Jacobsen, Leuven (BE); Ioannis Psarrakis, Lavrion-Attica (GR); Konstantinos Lioumis, Lavrion-Attica (GR)

(21) Appl. No.: 16/243,252

(22) Filed: Jan. 9, 2019

Related U.S. Application Data
(60) Provisional application No. 62/616,556, filed on Jan. 12, 2018.

(51) Int. Cl. A61K 31/573; A61K 9/00; A61K 9/08

(52) U.S. Cl. CPC A61K 31/573; A61K 9/08; A61K 9/0053

(57) ABSTRACT

Disclosed herein are pharmaceutical compositions for oral administration. The compositions are premixed aqueous suspensions of methylprednisolone and/or homogeneous aqueous suspensions of methylprednisolone. The pharmaceutical compositions include either about 2 mg/ml to about 4 mg/ml methylprednisolone or, alternatively, about 3 mg/ml to about 4 mg/ml methylprednisolone. The pharmaceutical compositions are exceptionally stable upon storage and are palatable.

### CROSS-REFERENCE TO RELATED APPLICATIONS

This application claims the benefit of U.S. Provisional Patent Application No. 62/616,556, which was filed on Jan. 12, 2018, the disclosure of which is incorporated by reference in its entirety herein.

### TECHNICAL FIELD

The present disclosure relates to a pharmaceutical suspension of methylprednisolone for oral administration that has acceptable taste characteristics and long term stability.

### BACKGROUND

Methylprednisolone is a corticosteroid. The chemical name for methylprednisolone is 11β,17,21-trihydroxy-6α-methylpregna-1,4-diene-3,20-dione and its olecular weight is 374.5. It has the following chemical structure:

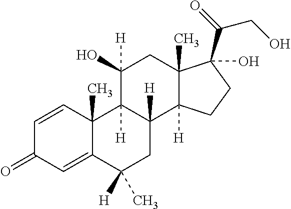

Methylprednisolone is used primarily as an anti-inflammatory or immunosuppressant agent in the treatment of a variety of diseases, including those of hematologic, allergic, inflammatory, neoplastic, and autoimmune origin. For example, methylprednisolone relieves inflammation (swelling, heat, redness, and pain) and is used to treat certain forms of arthritis; skin, blood, kidney, eye, thyroid, and intestinal disorders (e.g., colitis); severe allergies; and asthma. Methylprednisolone is also used to treat certain types of cancer. Unfortunately, methylprednisolone has a bitter taste and is very unpalatable. It is also practically insoluble in water.

Methylprednisolone acetate and sodium succinate salts are available as injectable compositions. DepoMedrol® (methylprednisolone acetate injection, suspension, 20 mg/mL, 40 mg/mL, and 80 mg/mL) is available for intramuscular, intra-articular, soft tissue or intralesional injection. DepoMedrol® (methylprednisolone acetate sterile aqueous suspension, 20 mg/mL, and 40 mg/mL) is available for intramuscular and intrasynovial injection in horses and dogs, and intramuscular injection in cats. Solu-Medrol (methylprednisolone sodium succinate for injection, 40 mg/mL, 125 mg/2 mL, 500 mg/4 mL, 500 mg/8 mL, 1 g/8 mL, 1 g/16 mL, and 2 g/30.6 mL) is available for intravenous or intramuscular administration.

Various strengths of methylprednisolone oral suspension, ranging from 2 mg/mL to 15 mg/mL, are available for animals from Wedgewood Pharmacy at WedgewoodPetRx.com.

Allen, Loyd V. Jr., International Journal of Pharmaceutical Compounding, Vol. 15, No. 4, July/August 2011 discloses another methylprednisolone oral suspension (4 mg/mL). However, use of this suspension is limited by lack of sufficient storage stability, i.e., it must be used within 14 days and must be stored in a refrigerator.

Pharmaceutical suspensions are particularly useful, for example, for pediatric and geriatric patients as they are convenient to swallow. Ready-to-use suspensions foster good patient compliance and acceptance in these patient populations. However, they often lack stability on storage. With the passage of time, the suspended drug particles tend to settle and even cake leading to inadequate or improper dosing.

There is a need for ready-to-use, stable, and efficiently taste-masked oral suspensions of methylprednisolone.

### SUMMARY

Disclosed herein is a premixed aqueous suspension for oral administration comprising about 2 mg/ml to about 4 mg/ml methylprednisolone. The suspension has a shelf life of least about 12 months when stored at about 25° C. and about 60% relative humidity.

Also disclosed herein is a premixed aqueous suspension for oral administration. The suspension comprises about 2 mg/ml to about 4 mg/ml methylprednisolone; a sweetener in an amount from about 0.1 wt % to about 2.0 wt %; a preservative in an amount from about 0.05 wt % to about 1.0 wt %; a suspending agent in an amount from about 0.05 wt % to about 2.0 wt %; a buffering agent in an amount from about 0.05 wt % to about 1.0 wt %; a flavoring agent in an amount from about 0.05 wt % to about 1.0 wt %; and a taste-masking agent in an amount from about 0.01 wt % to about 1.0 wt %. The suspension has a shelf life of least about 12 months when stored at about 25° C. and about 60% relative humidity.

In some embodiments, the suspensions disclosed herein comprise about 3 mg/ml to about 4 mg/ml methylprednisolone. In other embodiments, the suspensions disclosed herein comprise about 3.2 mg/ml methylprednisolone.

Also disclosed is a method of treating allergic states, dermatologic diseases, endocrine disorders, gastrointestinal diseases, hematologic disorders, neoplastic diseases, nervous system diseases, ophthalmic diseases, renal diseases, respiratory diseases, rheumatic disorders, trichinosis with neurologic or myocardial involvement, or tuberculous meningitis with subarachnoid block or impending block, comprising administering an effective amount of a premixed aqueous suspension of methylprednisolone for oral administration.

### BRIEF DESCRIPTION OF THE DRAWINGS

FIG. 1 provides the chromatogram supplied with methylprednisolone for system suitability CRS referenced in the European Pharmacopoeia monograph for methylprednisolone for identification of impurities.

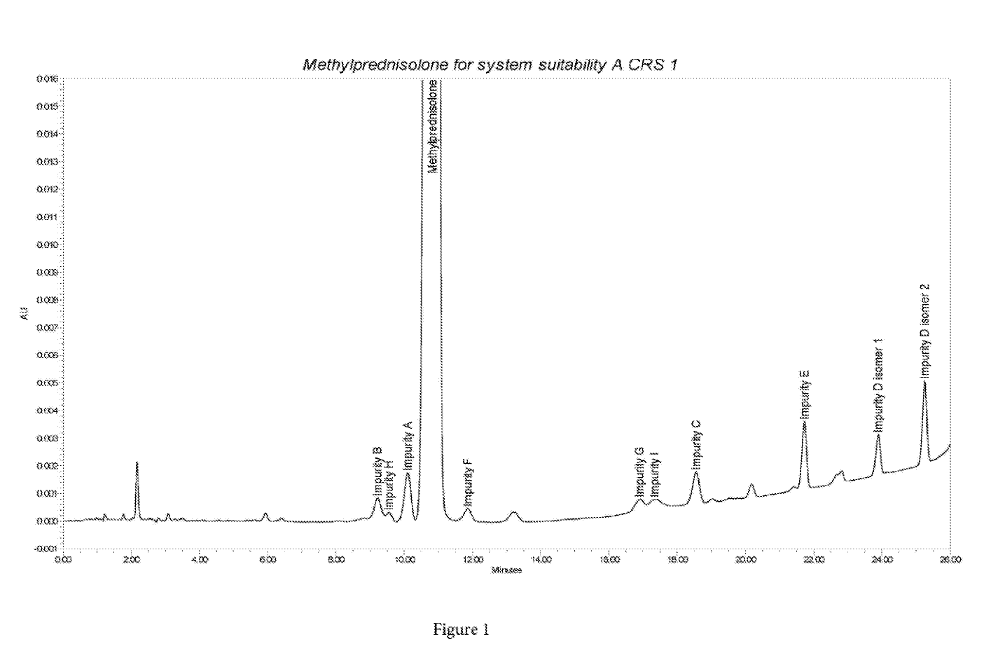

FIG. 2 is a table showing long term stability results for Suspension A (storage conditions: 25° C.+/−2° C., relative humidity=60+/−5%).

FIG. 3 is a table showing intermediate stability results for Suspension A (storage conditions: 30° C.+/−2° C., relative humidity=65+/−5%).

FIG. 4 is a table showing accelerated stability results for Suspension A (storage conditions: 40° C.+/−2° C., relative humidity=75+/−5%).

FIG. 5 is a table showing long term stability results for Suspension B (storage conditions: 25° C.+/−2° C., relative humidity=60+/−5%).

FIG. 6 is a table showing accelerated stability results for Suspension B (storage conditions: 40° C.+/−2° C., relative humidity=75+/−5%).

### DETAILED DESCRIPTION

The following detailed description is exemplary and explanatory and the following examples are intended to provide further explanation of the pharmaceutical compositions disclosed herein. Other features will be readily apparent to those skilled in the art from the following examples which are provided to show the advantageous stability of the pharmaceutical compositions. However, these examples are for illustrative purposes only.

The present disclosure relates to pharmaceutical compositions for oral administration. The pharmaceutical compositions are premixed aqueous suspensions of methylprednisolone that are stable upon storage. In some embodiments, the composition has a shelf life of least about 12 months when stored at about 25° C. and about 60% relative humidity. In some embodiments, the composition has a shelf life of at least about 24 months when stored at about 25° C. and about 60% relative humidity. In other embodiments, the composition has a shelf life of the at least about 36 months when stored at about 25° C. and about 60% relative humidity. As used herein and as generally understood in the art, “shelf life” refers to expiration date information submitted to a regulatory body responsible for approval of marketing the pharmaceutical composition. In the context of shelf life, “at least” with respect to a period of months means that the pharmaceutical composition may be labeled with an expiration date representing the last day of the specified time period, although it may retain stability properties thereafter.

In certain embodiments, the composition retains at least about 90% of the release amount of methylprednisolone after storage at about 25° C. and about 60% relative humidity for about six months. In some embodiments, the pharmaceutical composition retains at least about 90% of the release amount of methylprednisolone after storage at about 25° C. and about 60% relative humidity for about 12 months.

In other embodiments, the composition can retain at least about 95% of the release amount of methylprednisolone after storage at about 25° C. and about 60% relative humidity for about six months. In some embodiments, the composition can retain at least about 95% of the release amount of methylprednisolone after storage at about 25° C. and about 60% relative humidity for about 12 months.

In certain embodiments, the composition can retain from about 95% to about 105% of the release amount of methylprednisolone after storage at about 25° C. and about 60% relative humidity for about six months. In certain embodiments, the composition can retain from about 95% to about 105% of the release amount of methylprednisolone after storage at about 25° C. and about 60% relative humidity for about 12 months.

The present disclosure also relates to a pharmaceutical composition for oral administration comprising a homogeneous aqueous suspension. In embodiments of the pharmaceutical compositions disclosed herein, the composition is homogeneous after storage at about 25° C. and about 60% relative humidity for about one year. In some embodiments, the composition is homogeneous after storage at about 25° C. and about 60% relative humidity for about 24 months.

The premixed aqueous suspensions and homogeneous aqueous suspensions disclosed herein comprise either about 2 mg/ml to about 4 mg/ml or, alternatively, about 3 mg/ml to about 4 mg/ml methylprednisolone. For example, the premixed aqueous suspensions and homogeneous aqueous suspensions can comprise about 3.2 mg/ml methylprednisolone.

The pharmaceutical compositions disclosed herein can be conveniently prepared, stored, and administered. By virtue of the aqueous suspensions being premixed and/or homogeneous and stable upon storage, the pharmaceutical compositions disclosed herein can be prepared and stored in a ready-to-use dosage form. “Premixed” means that the dosage form is ready-to-use, i.e., it is manufactured and sold in a form that can be directly administered to patients without the need for any preparation steps, such as dilution, prior to administration. Children and geriatric patients, among others, who have difficulty swallowing oral solid dosage forms are likely to benefit from the suspensions described herein, thereby improving patient compliance and accordingly, treatment outcomes.

The pharmaceutical compositions disclosed herein can include one or more of: a sweetener, a preservative, a suspending agent, a buffering agent, a flavoring agent, and a taste-masking agent.

The premixed aqueous suspension and the homogeneous aqueous suspension can further comprise one or more of: a sweetener in an amount from about 0.1 wt % to about 2.0 wt %; a preservative in an amount from about 0.05 wt % to about 1.0 wt %; a suspending agent in an amount from about 0.05 wt % to about 2.0 wt %; a buffering agent in an amount from about 0.05 wt % to about 1.0 wt %; a flavoring agent in an amount from about 0.05 wt % to about 1.0 wt %; and a taste-masking agent in an amount from about 0.01 wt % to about 1.0 wt %. As used herein, the term “wt %” refers to weight/weight percentage.

The term “sweetener,” as used herein, refers to both caloric and non-caloric sweeteners. Exemplary sweeteners include acesulfame, alitame, arabinose, aspartame, cellobiose, cyclamate, dextrin, fructose, galactose, glucose, isomalt, lactose, maltodextrin, maltose, mannitol, mannose, monellin, monoammonium glycyrrhizinate, neohesperidin, neotame, oxylose, ribose, saccharin, sorbose, a steviol glycoside, sucralose, sucrose, sugar, pharmaceutically acceptable salts thereof, and combinations thereof. The term “steviol glycoside,” as used herein, refers to a natural sweetener derived from the leaves of the stevia plant and includes stevioside, steviolbioside, rubusoside, dulcoside A, rebaudioside A, rebaudioside B, rebaudioside C, rebaudioside D, rebaudioside E, and rebaudioside F. In some embodiments, the sweetener is selected from sucralose, saccharin sodium, and a combination thereof. In certain embodiments, the sweetener is a combination of sucralose and saccharin sodium.

The term “preservative,” as used herein, refers to any agent that is included in the suspension to prevent microbial contamination (e.g., yeast, mold, bacteria, etc.). Exemplary preservatives include benzalkonium chloride, benzethonium chloride, benzoic acid, benzyl alcohol, bronopol, butyl paraben and pharmaceutically acceptable salts thereof, cetyltrimethylammonium bromide, chlorhexidine, chlorobutanol, chlorocresol, ethyl alcohol, ethylenediaminetetraacetic acid, ethyl paraben and pharmaceutically acceptable salts thereof, imidurea, methyl paraben and pharmaceutically acceptable salts thereof, phenol, pharmaceutically acceptable phenylmurcuric salts, potassium sorbate, propylene glycol, propyl paraben and pharmaceutically acceptable salts thereof, sodium benzoate, sodium citrate, sodium propionate, sorbic acid, thimerosol, and combinations thereof. In some embodiments, the preservative is methyl paraben sodium.

The term “suspending agent,” as used herein, refers to any agent that helps keep methylprednisolone particles in suspension. Exemplary suspending agents include acacia, alginic acid, bentonite, a carbomer, carboxymethylcellulose calcium, carrageenan, colloidal silicon dioxide, crospovidone, dextrin, fumed silica, gelatin, guar gum, hydroxylethylcellulose, hydroxyethyl propylcellulose, hydroxylpropyl cellulose, hydroxypropyl methylcellulose, methylcellulose, magnesium aluminometasilicate, maltodextrin, microcrystalline cellulose, polydextrose, polyvinyl alcohol, povidone, propylene glycol alginate, sodium alginate, sodium carboxymethylcellulose, starch, tragacanth, xanthan gum, and combinations thereof. In certain embodiments, the preservative is xanthan gum.

The term “buffering agent,” as used herein, refers to any agent or combination of agents that resists changes in pH by the action of its acid-base conjugate components. Exemplary buffering agents include acetic acid, an acetate (e.g., sodium acetate), calcium carbonate, citric acid, disodium hydrogen phosphate, lactic acid, magnesium hydroxide, monosodium citrate, phosphoric acid, potassium citrate, sodium citrate, sodium hydrogen carbonate, sodium hydroxide, sodium phosphate, succinic acid, tartaric acid, and combinations thereof. In some embodiments, the buffering agent is selected from citric acid, sodium citrate, and a combination thereof. In certain embodiments, the buffering agent is a combination of citric acid and sodium citrate.

The term “flavoring agent,” as used herein, refers to an agent used to induce a flavor sensation in a human upon ingestion and includes both natural and synthetic flavoring agents. Exemplary flavoring agents include a banana flavoring agent, a black currant flavoring agent, a caramel flavoring agent, a cherry flavoring agent, a chocolate flavoring agent, a cream flavoring agent, a grenadine flavoring agent, a tutti frutti flavoring agent, a grape flavoring agent, a raspberry flavoring agent, a strawberry flavoring agent, a peppermint flavoring agent, and combinations thereof In some embodiments, the flavoring agent comprises peppermint. In certain embodiments, the flavoring agent is a peppermint flavoring agent. In some embodiments, the flavoring agent is a tutti frutti flavoring agent.

The term “taste-masking agent,” as used herein, refers to any agent or combination that potentiates, modifies or enhances the palatability of methylprednisolone, which is inherently bitter to the human palate. Flavoring agents and sweeteners can act as taste-masking agents.

Alcohols are exemplary taste masking agents. Additional exemplary taste-masking agents include cyclodextrins, maltodextrin, amino acids, gelatin, gelatinized starch, liposomes, lecithin, lecithin-like substances, salts, and combinations thereof. A “lecithin” is generally a phosphatide which occurs in egg yolk. As used herein, “lecithin-like substances” are phosphatides isolated from animals and plants other than the phosphatide lecithin that occurs in egg yolk. For example, “lecithin-like substances” include phosphatides isolated from soybean, liver, muscles, kidney, brain, nerves, blood, etc. As used herein, in reference to taste-making agents, exemplary salts are alkali metal bicarbonates such as sodium bicarbonate or glycyrrhizinate monoammonium. In some embodiments the taste-making agent is a combination of glycyrrhizinate monoammonium, sucralose, and maltodextrin. Suitable taste-masking agents are commercially available, for example, SC202039 masker flavor powder available from International Flavors and Fragrance's Inc., Magnasweet® 110, Magnasweet® 110F, Magnasweet® 110 2X, and Magnasweet® 110 3X.

As used herein, an aqueous suspension is “homogeneous” when each individual dose of the aqueous suspension is between 85% and 115% of the average dose when tested for homogeneity as set forth in the British Pharmacopoeia (2008), which is incorporated by reference in its entirety herein:

Homogeneity of Suspension
Allow a suitable volume of the oral suspension being examined to settle, undisturbed, for 24 hours. Shake the container for 30 seconds and accurately remove one dose (usually 5 to 10 ml) at a depth of 1 cm below the meniscus. Shake the container again for 10 seconds and remove another dose. Repeat this procedure until 10 doses of the suspension have been removed. Assay the 10 doses individually according to the method specified in the individual monograph.
The preparation complies with the test if each individual dose is between 85% and 115% of the average dose. The preparation fails to comply with the test if more than one individual dose is outside these limits or if one individual dose is outside the limits of 75% to 125% of the average content.

In some embodiments, the methylprednisolone is micronized. As used herein, the term “micronized” refers to methylprednisolone in the form of a fine powder having particles with diameters on the order of microns. In certain embodiments, the diameter of at least 90% of the micronized methylprednisolone is about 12 to about 15 micrometers. In embodiments, about 100% of the micronized methylprednisolone has a diameter less than about 30 micrometers, about 95% of the micronized methylprednisolone has a diameter less than about 15 micrometers, and about 50% of the micronized methylprednisolone has a diameter less than about 5 micrometers. When referring to micronized methylprednisolone herein, the particle size is measured according to the following method:
Apparatus: Malvern 2000 MU
Bump speed: 2500 rpm
Ultrasonic strength: 10.00
Particle Refraction index: 1.640
Result units: volume distribution
Size range: 0.02˜2000 μms
Obscuration:10˜20
Dispersant solution: water
Preparation of solution
1. Prepare 2% 6-sodiummetaphosphate solution:
Take about 2 g 6-sodium hexametaphosphate into a 100 ml iodimetric flask and dilute by 100 ml purified water.
2. Preparation of saturated solution:
Put about 1-2 g sample into a 1000 ml beaker and add 1000 ml purified water. Stir to obtain the saturated solution. Filter it by a suction flask to get a clear solution.
3. Preparation of suspension solution:
Put about 0.5-1 g sample into a beaker and add about 20 ml purified water to prepare a concentrated suspension solution.
Finally add 1-2 drops of 6-sodiummetaphosphate solution by burette, and dispense evenly.

The pH of the suspensions can vary. The pH of the can be, for example, about 4.0 to about 6.0. As another example, the pH can be about 5.0 to about 5.5.

Upon storage, methylprednisolone is subject to formation of impurities. As used herein, Impurities A-L refer to those impurities identified in the European Pharmacopoeia, Eighth Edition, Volume 2 (2013) monograph related to methylprednisolone (pp. 2748-2750), which is incorporated by reference in its entirety herein. Accordingly, Impurities A-L have the following meanings:

“Impurity A” refers to 17,21-dihydroxy-6α-methylpregna-1,4-diene-3,11,20-trione having the following structure:

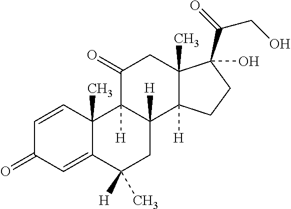

“Impurity B” refers to 11β,17,21,21-tetrahydroxy-6α-methylpregna-1,4-diene-3,20-dione having the following structure:

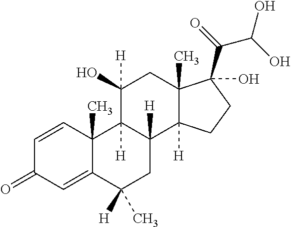

“Impurity C” refers to 11β-hydroxy-6α-methylandrosta-1,4-diene-3,17-dione having the following structure:

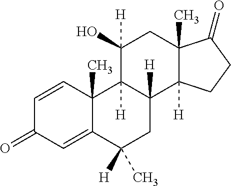

“Impurity D” refers to (EZ)-11β,20-dihydroxy-6α-methylpregna-1,4,17(20)triene-3,21-dione having the following structure:

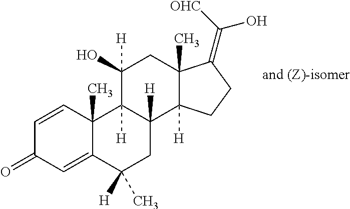

“Impurity E” refers to 11β-hydroxy-6α-methyl-3-oxoandrosta-1,4-diene-17β-carboxylic acid having the following structure:

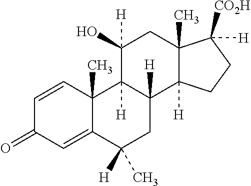

“Impurity F” refers to 11β,17,21-trlhydroxy-6α-methylpregn-4-ene-3,20-dione having the following structure:

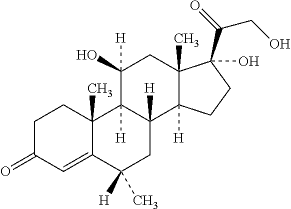

“Impurity G” refers to 17,21-dihydroxy-6α-methylpregna-1,4,9(11)-triene-3,20-dione having the following structure:

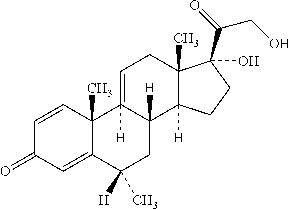

“Impurity H” refers to 11β,17,21-trihydroxy-6β methylpregna-1,4-diene-3,20-dione having the following structure:

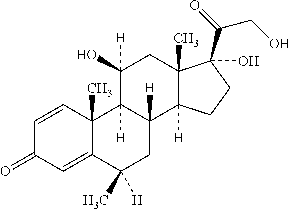

“Impurity I” refers to a compound having an unknown structure. It is identified as set forth in the European Pharmacopoeia, Eighth Edition, Volume 2 (2013) monograph related to methylprednisolone (pp. 2748-2750), which is incorporated by reference in its entirety herein.

“Impurity J” refers to 11β,17-dihydroxy-6α-methyl-3,20-dioxopregna-1,4-dien-21-yl acetate (methylprednisolone acetate) having the following structure:

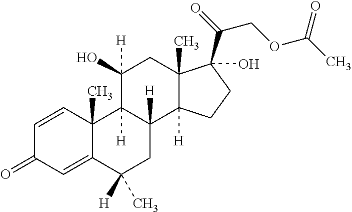

“Impurity K” refers to 11β,17,21-trihydroxypregna-1,4-diene-3,20-dione (prednisolone) having the following structure:

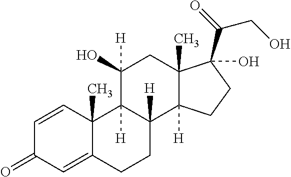

“Impurity L” refers to 11β,17-dihydroxy-6α-methylpregna-1,4-diene-3,20-dione having the following structure:

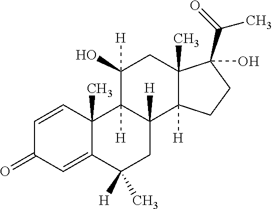

Impurities are identified as set forth in the European Pharmacopoeia, Eighth Edition, Volume 2 (2013) monograph related to methylprednisolone (pp. 2748-2750). With regard to impurity identification, this monograph specifically discloses:
Related substances. Liquid Chromatograph (2.2.29).
Solvent mixture: phosphoric acid R, acetonitrile R, water R (0.1:50:50 V/V/V)
Test solution. Dissolve 30.0 mg of the substance to be examined in the solvent mixture and dilute to 50.0 mL with the solvent mixture.
Reference solution (a). Dissolve 6 mg of methylprednisolone for system suitability CRS (containing impurities A, B, C, D, E, G and I) in the solvent mixture and dilute to 10.0 mL with the solvent mixture.
Reference solution (b). Dilute 1.0 mL of the test solution to 100.0 mL with the solvent mixture.
Reference solution (c). Dissolve 30.0 mg of methylprednisolone CRS in the solvent mixture and dilute to 50.0 mL with the solvent mixture.
Column:
size: l=0.15 m, Ø=4.6 mm;
stationary phase: end-capped octadecylsilyl silica gel for chromatrography R (3 μm);
temperature: 45° C.
Mobile phase:
mobile phase A: phosphoric acid R, tetrahydrofuran R, acetonitrile R, water R (0.1:1.5:10:90 V/V/V/V);
mobile phase B: phosphoric acid R, tetrahydrofuran R, acetonitrile R (0.1:1.5:100 V/V/V);

| Time | Mobile phase A | Mobile phase B |
|---|---|---|
| (min) | (percent V/V) | (percent V/V) |
| 0-14 | 83 | 17 |
| 14-30 | 83 → 52 | 17 → 48 |

Flow rate: 1.5 mL/min.
Detection: spectrophotometer at 247 nm.
Injection: 10 μL of the test solution and reference solutions (a) and (b).
Identification of impurities: use the chromatogram supplied with methylprednisolone for system suitability CRS and the chromatrogram obtained with reference solution (a) to identify the peaks due to impurities A, B, C, D, E, G, and I.
Relative retention with reference to methylprednisolone (retention time=about 12 min): impurity B=about 0.85; impurity H=about 0.8; impurity A=about 0.92; impurities G and I=about 1.54; impurity C=about 1.7; impurity E=about 1.9; impurity D (isomer 1)=about 2.10; impurity D (isomer 2)=about 2.2.
System suitability: reference solution (a):
resolution: minimum 1.7 between the peaks due to impurity A and methylprednisolone

Regarding impurity formation upon storage, in some embodiments, the pharmaceutical composition contains not more than about 0.50% of impurity D after storage for about 6 months at about 40° C. and about 75% relative humidity. Similarly, in some embodiments, the composition contains not more than about 0.30% of impurity G and impurity I combined after storage for about 6 months at about 40° C. and about 75% relative humidity.

Also provided herein is a method of treating allergic states, dermatologic diseases, endocrine disorders, gastrointestinal diseases, hematologic disorders, neoplastic diseases, nervous system diseases, ophthalmic diseases, renal diseases, respiratory diseases, rheumatic disorders, trichinosis with neurologic or myocardial involvement, or tuberculous meningitis with subarachnoid block or impending block, comprising administering an effective amount of a suspension disclosed herein.

As used herein, the term “effective amount” means the methylprednisolone dosage that provides the specific pharmacological response for which methylprednisolone is administered. An “effective amount” of methylprednisolone that is administered to a particular subject in a particular instance will not always be effective in treating the conditions/diseases described herein, even though such a dosage is deemed to be an “effective amount” by those of skill in the art.

Allergic states that can be treated by administration of the suspensions described herein include severe or incapacitating allergic conditions intractable to adequate trials of conventional treatment in asthma, atopic dermatitis, contact dermatitis, drug hypersensitivity reactions, seasonal or perennial allergic rhinitis, serum sickness, and transfusion reactions.

Dermatologic diseases that can be treated by administration of the suspensions described herein include bullous dermatitis herpetiformis, severe psoriasis, exfoliative erythroderma, exfoliative dermatitis, severe seborrheic dermatitis, mycosis fungoides, pemphigus, and severe erythema multiforme (Stevens-Johnson syndrome).

Endocrine disorders that can be treated by administration of the suspensions described herein include primary or secondary adrenocortical insufficiency, congenital adrenal hyperplasia, hypercalcemia associated with cancer, and nonsuppurative thyroiditis.

Gastrointestinal diseases that can be treated by administration of the suspensions described herein include regional enteritis and ulcerative colitis.

Hematologic disorders that can be treated by administration of the suspensions described herein include acquired (autoimmune) hemolytic anemia, congenital (erythroid) hypoplastic anemia (Diamond Blackfan anemia), pure red cell aplasia, idiopathic thrombocytopenia purpura, erythroblastopenia (RBC anemia) and secondary thrombocytopenia.

Neoplastic diseases that can be treated by administration of the suspensions described herein include leukemias and lymphomas.

Nervous system diseases that can be treated by administration of the suspensions described herein include acute exacerbations of multiple sclerosis; and cerebral edema associated with primary or metastatic brain tumor or craniotomy.

Ophthalmic diseases that can be treated by administration of the suspensions described herein include sympathetic ophthalmia, temporal arteritis, allergic corneal marginal ulcers, uveitis, diffuse posterior uveitis and choroiditis, keratitis, optic neuritis, herpes zoster ophthalmicus, allergic conjunctivitis, chorioretinitis, iritis and iridocyclitis, anterior segment inflammation and ocular inflammatory conditions unresponsive to topical corticosteroids.

Renal diseases that can be treated by administration of the suspensions described herein include idiopathic nephrotic syndrome and lupus erythematosus.

Respiratory diseases that can be treated by administration of the suspensions described herein include berylliosis, fulminating or disseminated pulmonary tuberculosis, idiopathic eosinophilic pneumonias, Loeffler's syndrome not manageable by other means, aspiration pneumonitis and symptomatic sarcoidosis.

Rheumatic disorders that can be treated by administration of the suspensions described herein include acute gouty arthritis, synovitis of osteoarthritis, post-traumatic osteoarthritis, acute nonspecific tenosynovitis, epicondylitis, acute rheumatic carditis, ankylosing spondylitis, psoriatic arthritis, rheumatoid arthritis, dermatomyositis, polymyositis, acute and subacute bursitis and systemic lupus erythematosus.

The subject to which the suspensions may be administered is generally a human. The human may be of any age, however, in certain embodiments, the human to whom the pharmaceutical composition is administered is a child or an infant. In various embodiments, the child or infant is less than 16 years old, less than 12 years old, less than 8 years old, less than 6 years old, less than 4 years old, or less than 2 years old. In other embodiments, the human to whom the pharmaceutical composition is administered is a geriatric subject. In various embodiments, the geriatric subject is at least 65 years old, at least 70 years old, or at least 75 years old.

### EXAMPLES

The results of International Council for Harmonisation of Technical Requirements for Pharmaceuticals for Human Use (ICH) stability studies performed on methylprednisole oral suspension (10 mg/5 ml and 16 mg/5 ml) packed in amber glass bottles are set forth below. One lab-trial batch of methylprednisolone oral suspension (16 mg/5 ml) (“Suspension A”) was tested for stability. Another lab-trial batch of methylprednisolone oral suspension (10 mg/5 ml) (“Suspension B”) was also tested for stability.

The testing of Suspension A was performed according to the following stability protocol:

| TESTING INTERVALS (in months) | | | | | | |
|---|---|---|---|---|---|---|
| 0 | 1 | 2 | 3 | 4 | 5 | 6 |

| | | | | | |
|---|---|---|---|---|---|
| CONDITION | | | | | |
| LONG TERM | X | X | X | X | X |
| 25° C. ± 2° C./60% RH ± 5% | | | | | |
| INTERMEDIATE | | X | X | X | X |
| 30° C. ± 2° C./65% RH ± 5% | | | | | |
| ACCELERATED | | X | X | X | X |
| 40° C. ± 2° C./75% RH ± 5% | | | | | |
| METHODS | | | | | |
| Homogeneity of suspension | X | | X | | X |
| Assay MTP | X | X | X | X | X |
| Assay methylparaben | X | X | X | X | X |
| Related Substances | X | X | X | X | X |
| Dissolution | X | | X | | X |
| XRD | X | | | | X |
| Antimicrobial Effectiveness | X | | | | |
| Test (AET) | | | | | |

The testing of Suspension B performed according to the following stability protocol:

| | TESTING INTERVALS (in months) | | | | | | |
|---|---|---|---|---|---|---|---|
| | 0 | 1 | 2 | 3 | 4 | 5 | 6 |
| CONDITION | | | | | | | |
| LONG TERM | X | X | X | X | X | X | X |
| 25° C. ± 2° C./60% RH ± 5% | | | | | | | |
| ACCELERATED | X | X | X | X | X | X | X |
| 40° C. ± 2° C./75% RH ± 5% | | | | | | | |
| METHODS | | | | | | | |
| Homogeneity of suspension | X | | | X | | | X |
| Assay MTP | X | X | X | X | X | X | X |
| Assay methylparaben | X | X | X | X | X | X | X |
| Related Substances | X | X | X | X | X | X | X |
| Dissolution | X | X | X | X | X | X | X |
| Antimicrobial Effectiveness | X | | | | | | |
| Test (AET) | | | | | | | |

The respective batches had the following compositions:

| | Suspension B | Suspension A |
|---|---|---|
| Methylprednisolone | 0.20 | 0.32 |
| Sucralose | 0.70 | 0.70 |
| Tutti Frutti 77919-33 Givaudan | 0.12 | |
| Peppermint flavor | | 0.12 |
| Masking flavor 4626 IFF | 0.10 | 0.10 |
| Methyl paraben sodium | 0.20 | 0.20 |
| Saccharin sodium | 0.20 | 0.20 |
| Xanthan gum FF | 0.23 | 0.23 |
| Citric acid anhydrous | 0.18 | 0.18 |
| Trisodium citrate | 0.20 | 0.20 |
| Purified water | 97.87 | 97.75 |
| Total (g) | 100.00 | 100.00 |

The analytical method for the related substances of methylprednisolone oral suspension was developed based on the related substances analytical method described in the European Pharmacopoeia, Eighth Edition, Volume 2 (2013) monograph related to methylprednisolone (pp. 2748-2750), which is incorporated by reference in its entirety herein. Impurity D: Impurity D is defined herein and in some embodiments is limited to 0.5%. Impurity A: Impurity A is defined herein and in some embodiments is limited to 0.3%. Impurities G and I: Impurity G and impurity I are defined herein and in some embodiments are limited to 0.3% collectively (i.e., the sum of impurities G and I is not more than 0.3%).

Impurities C, E and F: Impurities C, E and F are defined herein and in some embodiments are limited to 0.15%. This limit corresponds to the threshold for qualification of degradation products according to the ICH Topic Q 3 A (R2) Impurities in New Drug Substances, as well as the maximum daily dose (1g of the drug substance) in the drug product SPC (FI). The limits of imp. C, E and F are set according to the ICH Topic Q 3 B (R2) Impurities in New Drug Products, as well as the maximum daily dose in the drug product SPC (FI). Taking into account the above information: Identification threshold is 0.20%. Qualification threshold is 0.20%.

Any other impurity: Any other impurity includes any other detectable, potential, specified impurities (J, K and L), which are defined herein. Any other impurity also includes any unknown/unspecified impurities presented in the drug product. The limits are set according to the ICH Topic Q 3 B (R2) Impurities in New Drug Product, as well as the maximum daily dose in the drug product SPC (FI). Taking into account the above information: Identification threshold is 0.20%.

| Compound | RRt |
|---|---|
| EP imp. B | 0.85 |
| EP imp. H | 0.88 |
| EP imp. A | 0.94 |
| Methylprednisolone | 1.00 |
| EP imp. F | 1.09 |
| EP imp. G and I | 1.59 |
| EP imp. C | 1.77 |
| EP imp. E | 1.95 |
| EP imp. D (isomer 1) | 2.10 |
| EP imp. D (isomer 2) | 2.21 |

Homogeneity of the suspension was determined by the test set forth above from the British Pharmacopoeia (2008), which is incorporated by reference in its entirety herein.

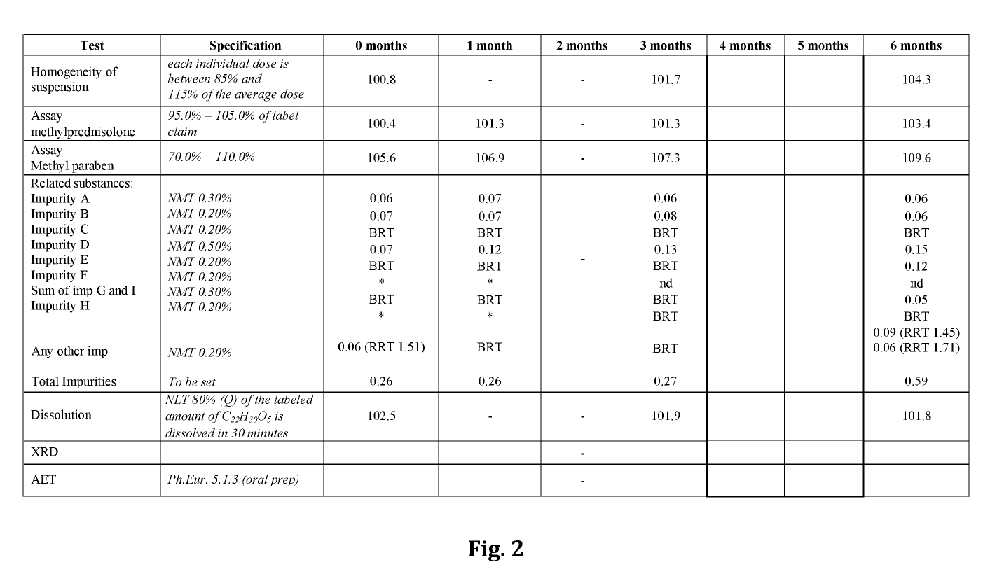

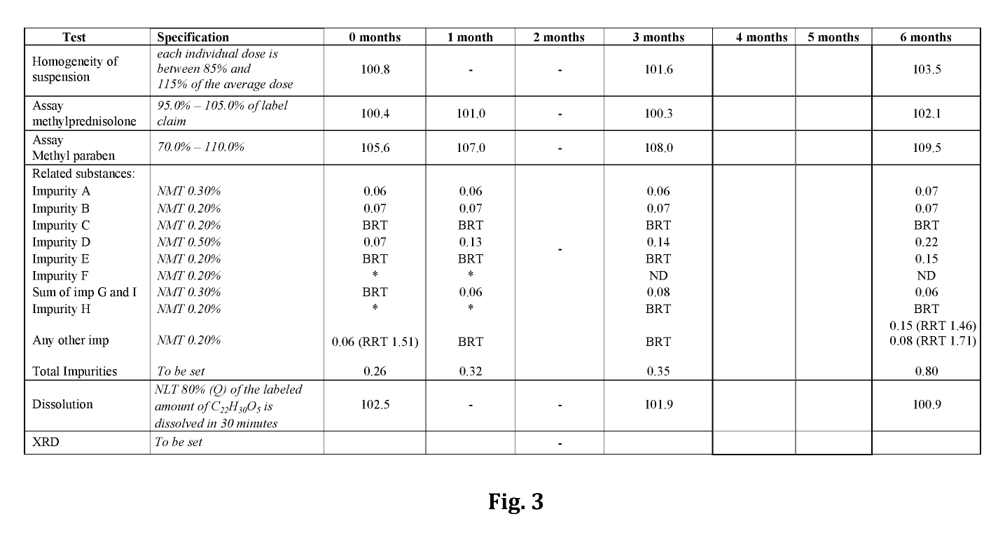

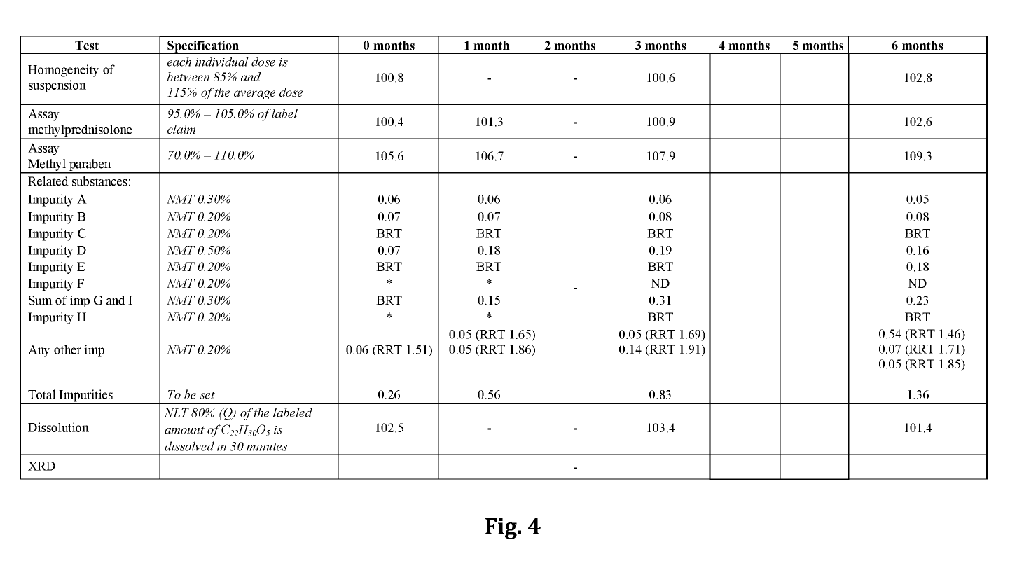

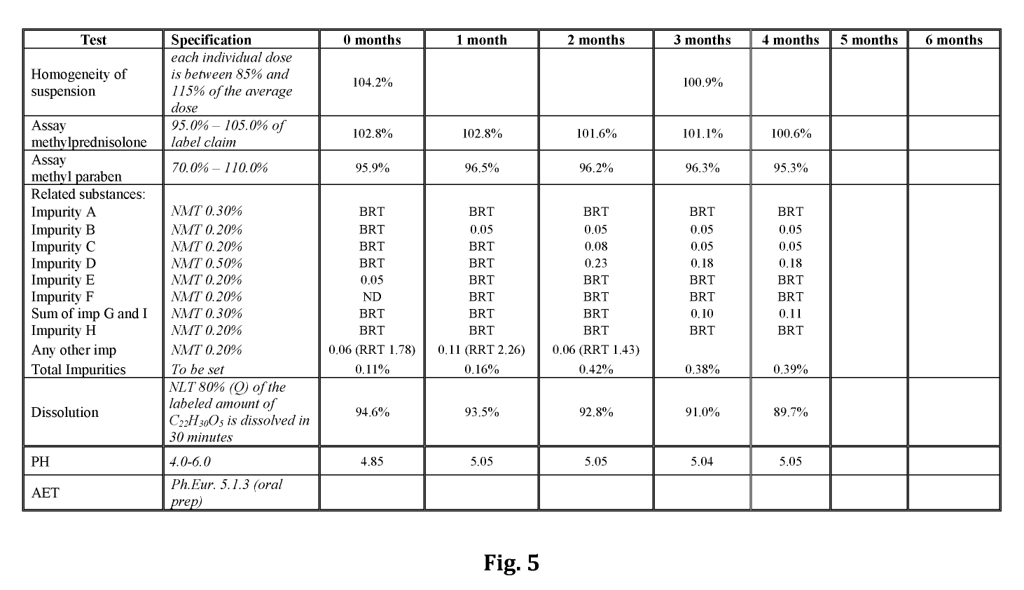

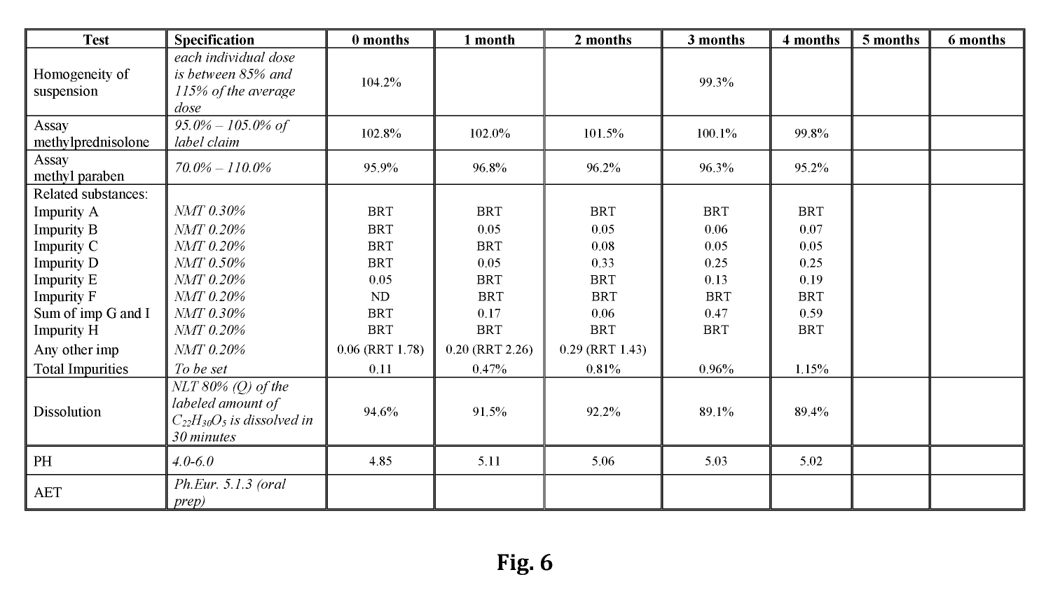

The stability results for the respective batches are shown in FIGS. 2 through 6.

Homogeneity of a 50 mL methylprednisolone suspension is determined by allowing the suspension to settle, undisturbed, for 24 hours. The container containing the 50 mL suspension is then shaken for 30 seconds. One 5 mL dose is removed at a depth of 1 cm below the meniscus. The container containing the remaining 45 mL of suspension is shaken again for 10 seconds and another 5 mL dose is removed. This procedure is repeated until all ten 5 mL doses of the suspension have been removed. The ten doses are assayed individually according to the following procedure:

Assay
Dissolve 0.100 g in alcohol R and dilute to 100.0 ml with the same solvent. Dilute 2.0 ml of the solution to 100.0 ml with alcohol R. Measure the absorbance (2.2.25) at the maximum at 243 nm.
Calculate the content of C₂₂H₃₀O₅ taking the specific absorbance to be 395.

The suspension is homogeneous if each 5 mL dose is between 85% and 115% of the average dose. The suspension is not homogeneous if more than one individual 5 mL dose is not between 85% and 115% of the average dose, or if one individual 5 mL dose is outside the limits of 75% to 125% of the average content.

What is claimed is:

1. A premixed aqueous suspension for oral administration comprising about 2 mg/ml to about 4 mg/ml methylprednisolone, wherein the suspension has a shelf life of least about 12 months when stored at about 25° C. and about 60% relative humidity.

2. The suspension of claim 1, comprising about 3 mg/ml to about 4 mg/ml methylprednisolone.

3. The suspension of claim 2, comprising about 3.2 mg/ml methylprednisolone.

4. The suspension of claim 1, wherein the suspension is homogenous.

5. The suspension of claim 1, wherein the methylprednisolone is micronized.

6. The suspension of claim 5, wherein the diameter of at least 90% of the micronized methylprednisolone is about 12 to about 15 micrometers.

7. The suspension of claim 4, wherein the suspension is homogeneous after storage at about 25° C. and about 60% relative humidity for about one year.

8. The suspension of claim 4, wherein the suspension is homogeneous after storage at about 25° C. and about 60% relative humidity for about 24 months.

9. The suspension of claim 1, wherein the suspension has a shelf life of at least about 24 months.

10. The suspension of claim 1, wherein the suspension has a shelf life of at least about 36 months.

11. The suspension of claim 1, wherein the pH of the suspension is about 4.0 to about 6.0.

12. The suspension of claim 11, wherein the pH of the suspension is about 5.0 to about 5.5.

13. A premixed aqueous suspension for oral administration comprising:
about 2 mg/ml to about 4 mg/ml methylprednisolone;
a sweetener in an amount from about 0.1 wt % to about 2.0 wt %;
a preservative in an amount from about 0.05 wt % to about 1.0 wt %;
a suspending agent in an amount from about 0.05 wt % to about 2.0 wt %;
a buffering agent in an amount from about 0.05 wt % to about 1.0 wt %;
a flavoring agent in an amount from about 0.05 wt % to about 1.0 wt %; and
a taste-masking agent in an amount from about 0.01 wt % to about 1.0 wt %,
wherein the suspension has a shelf life of least about 12 months when stored at about 25° C. and about 60% relative humidity.

14. The suspension of claim 13, comprising about 3.2 mg/ml methylprednisolone.

15. The suspension of claim 13, wherein the sweetener is selected from sucralose, saccharin sodium, and a combination thereof.

16. The suspension of claim 13, wherein the preservative is methyl paraben sodium.

17. The suspension of claim 13, wherein the suspending agent is xanthan gum.

18. The suspension of claim 13, wherein the buffering agent is selected from citric acid, sodium citrate, and a combination thereof.

19. The suspension of claim 13, wherein the flavoring agent is a tutti frutti flavoring agent.

20. The suspension of claim 13, wherein the taste-making agent comprises glycyrrhizinate monoammonium, sucralose, and maltodextrin.

21. A premixed aqueous suspension for oral administration comprising about 2 mg/ml to about 4 mg/ml methylprednisolone, wherein the suspension retains at least 90% of the release amount of methylprednisolone after storage at about 25° C. and about 60% relative humidity for about six months.

22. The suspension of claim 21 comprising about 3.2 mg/ml methylprednisolone.

23. The suspension of claim 21, wherein the suspension retains at least about 90% of the release amount of methylprednisolone after storage at about 25° C. and about 60% relative humidity for about 12 months.

24. The suspension of claim 21, wherein the suspension contains not more than about 0.50% of impurity D after storage for about 6 months at about 40° C. and about 75% relative humidity.

25. The suspension of claim 21, wherein the suspension contains not more than about 0.30% of impurity G and impurity I combined after storage for 6 months at about 40° C. and about 75% relative humidity.
